(19) United States
(12) Patent Application Publication
(10) Pub. No.: US 2009/0098213 A1
(43) Pub. Date: Apr. 16, 2009

(54) PAIN RELIEF AND SOOTHING CREAM AND METHOD FOR MAKING SAME

(76) Inventor: Huynh D. Tran, Santa Ana, CA (US)

Correspondence Address: FULWIDER PATTON LLP, 6060 CENTER DRIVE, 10TH FLOOR, LOS ANGELES, CA, 90045, US

(21) Appl. No.: 11/870,625

(22) Filed: Oct. 11, 2007

(51) Int. Cl. A61K 31/7008; A61K 31/05; A61K 31/10; A61K 31/125; A61K 31/192; A61K 36/282; A61K 36/54; A61K 36/899; A61P 17/00; A61P 29/00; A61K 36/906; A61K 36/61; A61K 36/48; A61K 35/12; A61K 31/195; A61K 31/245; A61K 31/4174

(52) U.S. Cl. 424/526; 424/739; 424/740; 424/742; 424/750; 424/756; 424/757; 514/396; 514/537; 514/567; 514/568; 514/62; 514/675; 514/711; 514/731

(57) ABSTRACT

emollient and methyl salicylate, where said methyl salicylate comprises between 10 and 30 percent by weight of said cream; a skin stimulant comprising menthol crystals and two or more reactants selected from a group of camphor, cinnamomum cassia, diclofenac, eucalyptus, and ginger to cause a hot and cool stimulus on an epidermal surface; a pain reliever comprising two or more agents selected from a group of benzocaine, emu oil, eucalyptus oil, methylsulfonylmethane and glucosamine; and an inflammatory comprising two or more agents selected from a group of artemisia vulgaris, amica Montana flower extract, licorice root extract, oat extract, and thyme oil.

### BACKGROUND OF THE INVENTION

The present invention relates generally to pain relief balms, and more particularly to a pain relief and soothing cream and method for making same.

Chronic pain from sore muscles and body aches affect millions of people each day. By one estimate, over thirty percent of the population have at least some pain or soreness on a daily basis. Chronic pain can lead to depression, reduced productivity, sleeplessness, and a general lower quality of life. In fact, several billion dollars of lost revenue in the form of missed work days, medical costs, and ineffective treatments can be attributed to pain and the pursuit of relief. Some pain is the result of injuries, where as other types of pain occurs from a degenerative condition or illness. Pain can also result from repetitive activities such as driving, typing, or lifting. Muscle injury or damage can also present as chronic pain, and can further indicate nerve damage or trauma.

Back pain, and particularly lower back pain, is a particularly disabilitating phenomenon that affects many people, particularly those of middle age or older. The lower back bears the weight of the upper body and is subject to large stresses when bending, twisting, or lifting. Lower back pain is usually the result of inflammation or damage to the ligaments, tendons, muscles, or discs of the skeletal structure. The most common causes of upper back pain related to tightness of flexibility of neck muscles in 3 areas: upper, middle and lower fibers, also called trapezius muscles on top of the shoulders. It cause significant discomfort to the people who work at the office, desks and computers for a long period of time due to sore muscles. The pain can manifest itself as a sharp piercing pain or a dull, burning sensation referred to as muscle ache.

Known back pain remedies are probably as old as the history of man, and yet the number of pain sufferers speaks to the effectiveness of present solutions. Anti-inflammatory drugs and strong pain killers or muscle relaxants may be prescribed for a short period of time for temporary relief, but are not satisfactory long term solutions. In sum, the prior art lacks a truly effective pain remedy that is safe and cost effective without creating dependency or impairing other functions.

### SUMMARY OF THE INVENTION

The present invention is a combination of an aqueous base having a thickening emollient and methyl salicylate, where said methyl salicylate comprises between 10 and 30 percent by weight of said cream; a skin stimulant comprising menthol crystals and two or more reactants selected from a group of camphor, cinnamomum cassia, diclofenac, eucalyptus, and ginger to cause a hot and cool stimulus on an epidermal surface; a pain reliever comprising two or more agents selected from a group of benzocaine, emu oil, and eucalyptus oil; and an inflammatory comprising two or more agents selected from a group of artemisia vulgaris, amica Montana flower extract, licorice root extract, oat extract, diclofenac, and thyme oil.

A second aspect of the present invention is a method for making a pain relief and soothing cream comprising the steps of combining in a first mixing tank a first mixture comprising water, preservatives, and a thickening agent, where the first mixture has a pH of between 3.5 and 4.5; combining in a second mixing tank a second mixture comprising a thickening agent, an antiseptic ingredient, an antifungal ingredient, an inflammatory ingredient, an anesthetic ingredient, a pain relieving ingredient, menthol, and camphor, where said second mixture has a pH of between 7.0 and 8.0; combining in a third mixing tank a third mixture comprising water, a pH adjuster, and an emulsifier agent, where the third mixture has a pH of between 11.5 and 12.5; combining the first mixture with the second mixture and allow to clarify, where the pH of the combination of the first mixture and the second mixture is between 3.5 and 4.5; and combining the third mixture to the combination of the first and second mixture and mix thoroughly to form a cream, where the cream has a pH of 6.5.

### DETAILED DESCRIPTION OF THE PREFERRED EMBODIMENTS

The present invention is a high potency, fast-acting pain relief and soothing cream that provides long-lasting relief to sore and tired muscles. The cream is formulated with selected arthritis, pain, and soothing topical analgesics, using natural ingredients in a unique liquid base. The cream provides a combination of cooling and heat sensations that increase circulating and promote faster healing in addition to relief from aching and stiff muscles. The cream is particularly effective in relaxing the trapezius muscles of the upper back and relieving aches associated with this muscle group of upper, middle and lower fibers which has several functions as to rotate and move shoulder blade up and down, in and out, to rotate and bend the neck in the different directions, the trapezious muscle respond very well to massage with this pain reliever and soothing cream. The cream has also been shown effective in the treatment of arthritis in elderly patients, tendonitis, neuralgia, and sports-related injuries.

The present invention is a unique combination of ingredients unlike that found in any other pain cream. The invention is preferably created in three parts, and then mixed according to the following steps. The first part, mixed in a first mixing chamber, is a combination of 47.35% water (where all percentages are by weight of total compound), 0.15% Dimethylol Dimenthul (DMDM) Hydantoin—Iodopropynyl Butylcarbamate, 0.1% Tetrasodium EDTA, and 0.65% Carbomer such as Carbopol Ultrez-10 (or Acrymer-10). DMDM Hydantoin—Todopropynyl Butylcarbamate is a preservative with moderate sensitizing potential. Tetrasodium EDTA is also a preservative as well as a sequestering and chelating agent. Carbomer is a thickening or suspending agent and an emulsion stabilizer. In this first part, the water is placed into a process mixing tank with a stirring mechanism on, whereupon the Dimethylol Dimenthyl (DMDM) Hydantoin—Iodopropynyl Butylcarbamate and Tetrasodium EDTA are added while stirring for 30 minutes. The stirring mechanism is then turned down to a very slow speed when adding the Carbomer Powder, remembering to use care when handling this powder to thicken the mixture. The stirring mechanism is started slowly to wet the powder by water, followed by increasing speed for 120 minutes or until it is fully dispersed. Once the mixture is fully thickened, the pH is tested to ensure that it has a value between 3.5 and 4.5.

The second part brings together a plurality of ingredients in a second mixing tank. First, 3.5% Hydrogenated Polyisobutene (Panalane L-14E) is a versatile pure hydrocarbon based emollient that is non-comedegenic, non-irritating, and stable over a wide range of temperature, pH and oxidation. It's an excellent moisturizer, a thickening agent and emollient that coats and protects the skin to prevent water loss, is combined with 12% methyl salycilate (wintergreen oil), an external antiseptic with anti-fungal properties, in a stainless steel mixing tank. The stirring mechanism is activated and added 0.05% diclofenac, an anti-inflammatory agent that also acts to relieve pain, and 0.15% Benzocaine, a topical anesthetic used in the treatment of dermatitis and sun burn. 0.05% Methylsulfonylmethane and 0.10% glucosamine, a combination used to treat joint pain and arthritis, is added to the second part mixture. This combination is stirred 30 minutes, whereupon the following ingredients are added: 1). 0.5% Oat extract; 2). 2% Artemisia Vulgaris, an algae extract rich in amino acid, polypeptides, and cell membrane carbohydrates with mugwort extract for relief from redness, irritated skin, and anti-stinging; 3). 1.2% Arnica Montana Flower extract, a plant with cooling properties and promotes cell renewal, the extract's active ingredient include sesquiterpene lactones used as counter-irritants and circulation enhancer as well as reducing swelling; 4). 0.2% Ginger Root extract, an analgesic systemic stimulating and producing a warming effect on the skin indicating temporary increase of peripheral blood flow to help reduce swelling and reduce stiffness of muscles and joints; 5). 3% Menthol crystal, a natural antipyretic (fever reducer) and antiseptic that produces a cooling effect on the skin; 6). 1% Camphor, an antimicrobital substance that is readily absorbed through the skin and produces a cooling effect similar to menthol with anesthetic qualities; and 7).). 4% Cinnamomun Cassia Leaf Extract, an essential oil having aromatic properties and a warming effect on the skin. This combination is stirred for thirty additional minutes. After thorough mixing, 8). 0.25% Licorice Root extract (stearyl glycyrrhetinate), a natural anti-inflammatory, reduce puffiness, irritation, anti-edema substance is mixed with 9). 0.10% Thyme extract, an essential oil extracted from the leaves of the Thymus Vulgaris plant that is known to have antibacterial, deodorizing, fungicidal, and circulation stimulating properties. 10). 0.30% Emu Oil, a skin emollient used in the treatment of bums and skin disorders, is added to the mixture along with 11). 0.50% Eucalyptus extract, an anti-spasmodic, disinfectant, and blood circulation promoting agent, along with 0.50% mineral and 0.05% Clotrimazole. The mineral oil helps improve the epidermal barrier function by providing a protective layer, and Clotrimazole is effective against a variety of fungi, a topical treatment of Athlete's foot, jock itch, and ringworm. This mixture is stirred at least sixty minutes or until it is fully dispersed, whereupon the stirring mechanism is deactivated. This second part is tested to ensure that the pH value is between 7.0-8.0.

In the third part, 25% water is mixed with 0.05% sodium hydroxide, a pH adjuster, and 0.60% triethanolamine, another pH adjuster, into a separate tank. A stirring mechanism is activated for fifteen minutes, whereupon 0.25% Methocel 40-202 or Hypromellose, two thickening and emulsifying agents that prevent separation of the mixture, is added to the solution. This mixture is continuously stirred at a high speed for one hundred twenty minutes or until it is fully dispersed and clear, whereupon the stirring mechanism is deactivated. This mixture is tested to ensure that the pH value is between 11.5 and 12.5.

Return to the first part tank and reactivate the stirring mechanism for thirty minutes, and then add the second part into the first part while stirring and increasing speed. The combination of the first part and the second part will thicken and clarify, as stirring is maintained for an additional one hundred and twenty minutes. The combination of the first part and the second part is tested to ensure that the solution has a pH value of between 3.5 and 4.5.

The first and second parts mixture is then combined with the third part mixture to complete the process. In the tank containing the first and second parts, the stirring mechanism is activated on high speed while the ingredients of the third part are slowly added to the tank. A high speed is necessary to ensure complete mixing as thickening is already present and accelerates in the first minute of introduction. The combination is mixed continuously for at least one hundred twenty minutes or until fully dispersed and uniform. Finally, the combination of first, second, and third parts is tested to ensure that the pH value is 6.5.

Where the final product requires a pH adjustment, the following steps are taken. To increase pH, add Sodium Hydroxide 20% solution to bring pH up higher to 6.5. To decrease pH, add Triethalolamine TEA to bring pH lower to 6.5. The final step is to cover the processed tank and let it sit overnight to completely gel and stabilize, and then perform quality control as necessary.

I claim:

1. A pain relief and soothing cream comprising:
an aqueous base having a thickening emollient and methyl salicylate, where said methyl salicylate comprises between 10 and 30 percent by weight of said cream;
a skin stimulant comprising menthol crystals and two or more reactants selected from a group of camphor, cinnamomum cassia, diclofenac, eucalyptus, and ginger to cause a hot and cool stimulus on an epidermal surface;
a pain reliever and soothing cream comprising two or more agents selected from a group of benzocaine, emu oil, eucalyptus oil, methylsulfonylmethane, and glucosamine; and
an inflammatory comprising two or more agents selected from a group of artemisia vulgaris, amica Montana flower extract, licorice root extract, oat extract, diclofenac and thyme oil.

2. A method for making a pain relief and soothing cream comprising the steps of:
combining in a first mixing tank a first mixture comprising water, preservatives, and a thickening agent, where the first mixture has a pH of between 3.5 and 4.5;
combining in a second mixing tank a second mixture comprising a thickening agent, an antiseptic ingredient, an inflammatory ingredient, an anesthetic ingredient, a pain relieving ingredient, menthol, and camphor, where said second mixture has a pH of between 7.0 and 8.0;
combining in a third mixing tank a third mixture comprising water, a pH adjuster, and an emulsifier agent, where the third mixture has a pH of between 11.5 and 12.5;
combining the first mixture with the second mixture and allow to clarify, where the pH of the combination of the first mixture and the second mixture is between 3.5 and 4.5; and
combining the third mixture to the combination of the first and second mixture and mix thoroughly to form a cream, where the cream has a pH of 6.5.

3. The method for making a pain relief and soothing cream of claim 2, where the preservative is selected from a group comprising dimethylol dimethyl hydantoin and tetrasodium EDTA.

4. The method for making a pain relief and soothing cream of claim 2, where the thickening agent in the first mixture is carbomer powder.

5. The method for making a pain relief and soothing cream of claim 2, where the thickening agent in the second mixture is hydrogenated Polyisobutene.

6. The method for making a pain relief and soothing cream of claim 2, where the antiseptic ingredient is methyl salycilate.

7. The method for making a pain relief and soothing cream of claim 2, where the inflammatory ingredient is diclofenac sodium.

8. The method for making a pain relief and soothing cream of claim 2, where the inflammatory ingredient is Arnica Montana Flower extract.

9. The method for making a pain relief and soothing cream of claim 2, where the inflammation ingredient is ginger root extract.

10. The method for making a pain relief and soothing cream of claim 2 wherein the step of combining in a second mixing tank includes mixing cinnamomum cassia leaf oil.

11. The method for making a pain relief and soothing cream of claim 2 wherein the step of combining in a second mixing tank includes mixing licorice root extract.

12. The method for making a pain relief and soothing cream of claim 2 wherein the step of combining in a second mixing tank includes mixing thyme extract oil.

13. The method for making a pain relief and soothing cream of claim 2 wherein the step of combining in a second mixing tank includes mixing emu oil.

14. The method for making a pain relief and soothing cream of claim 2 wherein the step of combining in a second mixing tank includes mixing eucalyptus oil.

15. The method for making a pain relief and soothing cream of claim 2 wherein the step of combining in a second mixing tank includes mixing mineral oil.

16. The method for making a pain relief and soothing cream of claim 2 wherein the step of combining in a second mixing tank includes mixing oat extract.

17. A pain relief and soothing cream comprising:
a first mixture comprising water, preservatives, and a thickening agent, where the first mixture has a pH of between 3.5 and 4.5;
a second mixture comprising a thickening agent, an antiseptic ingredient, an inflammatory ingredient, an anesthetic ingredient, a pain relieving ingredient, menthol, and camphor, where said second mixture has a pH of between 7.0 and 8.0;
a third mixture comprising water, a pH adjuster, and an emulsifier agent, where the third mixture has a pH of between 11.5 and 12.5;
wherein the first mixture and the second mixture are combined and allow to clarify, wherein the third mixture is combined therewith to form a cream having a pH of 6.5.

18. The pain relief and soothing cream of claim 17, where the preservative is selected from a group comprising dimethylol dimethyl hydantoin and tetrasodium EDTA.

19. The pain relief and soothing cream of claim 17, where the thickening agent in the first mixture is carbomer powder.

20. The pain relief and soothing cream of claim 17, where the thicken agent in the second mixture is hydrogenated polyisobutene.

21. The pain relief and soothing cream of claim 17, where the antiseptic ingredient is methyl salycilate.

22. The pain relief and soothing cream of claim 17, where the inflammatory ingredient is diclofenac sodium.

23. The pain relief and soothing ream of claim 17, where the inflammatory ingredient is Arnica Montana Flower extract.

24. The pain relief and soothing cream of claim 17, where the inflammatory ingredient is ginger root extract.

25. The pain relief and soothing cream of claim 17 wherein the second mixture includes cinnamomum cassia leaf oil.

26. The pain relief and soothing cream of claim 17 wherein the second mixture includes licorice root extract.

27. The pain relief and soothing cream of claim 17 wherein the second mixture includes thyme extract oil.

28. The pain relief and soothing cream of claim 17 wherein the second mixture includes emu oil.

29. The pain relief and soothing cream of claim 17 wherein the second mixture includes eucalyptus oil.

30. The pain relief and soothing cream of claim 17 wherein the second mixture includes mineral oil.

31. The pain relief and soothing cream of claim 17 wherein the second mixture includes clotrimazole.
